(19) United States
(12) Patent Application Publication
(10) Pub. No.: US 2010/0087788 A1
(43) Pub. Date: Apr. 8, 2010

(54) Anti-infective alcohol catheter solution with chlorhexidine treated catheter

(76) Inventors: Joel Rosenblatt, Pottstown, PA (US); Daniel J. Spangler, Wernersville, PA (US); Shanna Moss, Limerick, PA (US)

Correspondence Address: AMSTER, ROTHSTEIN & EBENSTEIN LLP, 90 PARK AVENUE, NEW YORK, NY, 10016, US

(21) Appl. No.: 12/311,647

(22) PCT Filed: Oct. 12, 2007
(86) PCT No.: PCT/US07/21835

§ 371 Date: Dec. 8, 2009

Related U.S. Application Data
(60) Provisional application No. 60/852,630, filed on Oct. 18, 2006.

(51) Int. Cl. A61L 15/46; A61L 2/18

(52) U.S. Cl. 604/265; 422/28

(57) ABSTRACT

Implantable catheters treated with chlorhexidine and methods for disinfecting the catheters with alcohol are provided.

### CROSS-REFERENCE TO RELATED APPLICATION

This application claims priority of U.S. Provisional Patent Application No. 60/852,630, filed on Oct. 18, 2006, the content of which is incorporated by reference.

### FIELD OF THE INVENTION

The present invention relates to medical devices, especially catheters such as intravenous catheters, with chlorhexidine (and/or its salts) incorporated in the device either as a coating or bulk distributed, used in synergistic combination with an alcohol lock solution to inhibit attachment and/or growth of microorganisms, thereby preventing device-related infection.

### BACKGROUND OF THE INVENTION

Various publications are referred to throughout this application. Full citations for these references may be found at the end of the specification immediately preceding the claims. The disclosures of these publications are hereby incorporated by reference in their entireties into the subject application to more fully describe the art to which the subject application pertains.

Intravenous catheters, when placed in the human body, serve as an attachment point for microorganisms, leading to biofilm formation and infection. Infection of the catheter hub and catheter-related blood stream infections are major complications for patients with indwelling catheters (e.g., Safdar and Maki 2003; Saint et al. 2000).

The antimicrobial activity of ethyl alcohol (ethanol) as well as other alcohols is well known. Isopropyl alcohol at a concentration of 60-70% is widely used as an antimicrobial agent for sanitization of surfaces and skin. A concentration of 10% ethyl alcohol inhibits the growth of most microorganisms, while concentrations of 40% and higher are generally considered bactericidal (Sissons et al. 1996).

Antimicrobial lock solutions have been used to address luminal introduction of microorganisms to patients' blood stream. The use of ethanol as a lock solution is known (Ball et al. 2003; Dannenberg et al. 2003; Metcalf et al. 2004; University of Wisconsin News Release, Aug. 10, 2005). Polymers commonly utilized to produce intravascular devices have been shown to be compatible with 70% ethyl alcohol (Crnich et al. 2005); however, not all polymers are compatible. The addition of other antimicrobial agents to lock solutions of lower alcohols, including ethyl alcohol, has also been described. A catheter hub containing an antiseptic chamber filled with 3% iodinated alcohol has been shown to significantly reduce the rate of catheter-related blood stream infections, when compared with a standard hub model (Segura et al. 1996): Finch et al. disclose in U.S. Pat. Nos. 6,679,870 and 6,685,694 the addition of triclosan or taurolidine to catheter lock solutions of lower alcohols, including ethyl alcohol. The addition of antibiotics to catheter lock solutions has been described as an infection prevention (prophylaxis) and treatment approach (Bestul et al. 2005; O'Grady et al. 2002).

The antimicrobial activity of chlorhexidine and its salts is known (Denton 1991). One approach to help prevent device-related infection is the incorporation of chlorhexidine in a coating or bulk distributing throughout the device (U.S. Pat. Nos. 5,451,424, 5,707,366, and 6,261,271).

Fox et al. disclose in U.S. Pat. No. 5,019,096 a method of preparing an infection-resistant medical device containing antimicrobial agents, especially a synergistic combination of a silver salt and chlorhexidine (or its salts). This patent also discloses medical devices with the synergistic composition.

Alcohol and chlorhexidine have been combined in solution. Viamonte et al. in U.S. Pat. No. 6,869,623 disclose a non-toxic mucosal disinfectant for topical application in the nose having a composition of 91% isopropyl alcohol of at least 50% by weight, sesame and lemon oils, aloe, and chlorhexidine gluconate as an optional component. Osborne et al. disclose the combination of chlorhexidine gluconate (CHG), SDA-3 ethyl alcohol and cetyl lactate producing a highly effective topical antimicrobial cleanser having immediate, persistent and residual bactericidal activity (U.S. Pat. Nos. 5,776,430 and 5,906,808, PCT International Publication No. WO 95/12395). Igarashi et al. in U.S. Pat. No. 5,800,827 disclose a disinfectant composition containing ethyl alcohol with a concentration of not lower than 50% by weight chlorhexidine and an organic acid. Peters discloses in U.S. Pat. No. 5,753,246 a sanitation kit having a packaged germicidal towelette for single-use wiping of the hands of a user, the germicide being a chlorhexidine alcohol solution for providing broad-spectrum disinfecting activity to the towelette. In U.S. Pat. No. 5,443,385 Overmyer describes a method of disinfecting and lubricating a discrete dental-medical device which involves immersing the device in a water-alcohol-glycerin-chlorhexidine solution. Kihara et al. in U.S. Pat. No. 5,017,617 disclose a disinfectant composition for medical use that has satisfactory bactericidal effects and that rarely causes skin damage even when used frequently. The composition comprises ethanol used for disinfection, a bactericidal agent such as chlorhexidine digluconate, and an emollient. A 2% solution of chlorhexidine is commonly utilized for cutaneous antisepsis at the time of intravascular device insertions, and an FDA approved commercial product utilized in conjunction with vascular access contains 2% chlorhexidine gluconate in 70% isopropyl alcohol (Crnich et al. 2005).

Despite these advances in combating infection, improved approaches are needed to prevent infections of implantable medical devices such as catheters.

### SUMMARY OF THE INVENTION

The present invention is based on the discovery of a synergistic relationship between chlorhexidine and alcohol to combat infection of implantable medical devices such as catheters with reduced risk of toxicity.

The invention provides implantable catheters comprising a lumen that is at least partly filled with a solution comprising alcohol, wherein the catheter is impregnated with chlorhexidine and/or the inner surface of the lumen is coated with chlorhexidine.

The invention also provides implantable catheters that can be disinfected in vivo with alcohol, wherein the catheter is impregnated with chlorhexidine and/or the catheter comprises an inner lumen that is coated with chlorhexidine, and wherein chlorhexidine is present in the catheter in a concentration that is subinhibitory for attachment or growth of microorganisms on the catheter.

The invention further provides methods of disinfecting or preventing infection of a catheter implanted in a subject, the method comprising flushing the inner lumen of the catheter with a solution comprising alcohol, where the implanted catheter is impregnated with chlorhexidine and/or the inner lumen is coated with chlorhexidine.

### BRIEF DESCRIPTION OF THE DRAWINGS

FIG. 1. Picture of enhanced Zone of Inhibition (ZOI) against Candida albicans for catheter segments impregnated with chlorhexidine base in the presence of 5% ethanol (left dish) or no ethanol or other alcohol (right dish) at 24 hours incubation. The presence of 5% ethanol increases the ZOI.

FIG. 2. Release curves of chlorhexidine from a catheter showing that chlorhexidine diacetate releases faster into a 25% ethanol lock solution than does chlorhexidine base. CHA—chlorhexidine diacetate; CHX—chlorhexidine base CHX.

### DETAILED DESCRIPTION OF THE INVENTION

The invention provides an implantable catheter comprising a lumen that is at least partly filled with a solution comprising alcohol, wherein at least an inner surface of the lumen is impregnated or coated with chlorhexidine.

The chlorhexidine can be present in the catheter in a concentration that, in the absence of alcohol, is subinhibitory for attachment or growth of microorganisms on the catheter, and/or alcohol can be present in a concentration that, in the absence of chlorhexidine, is subinhibitory for attachment or growth of microorganisms on the catheter. As used herein, a “subinhibitory” concentration of chlorhexidine or alcohol is a concentration that is below the concentration required to prevent or reduce attachment or growth of microorganisms on an implantable catheter. Minimum inhibitory concentration can be determined, for example, as set forth herein in the Examples. The chlorhexidine and/or alcohol can be present, for example, in a concentration that is subinhibitory for attachment or growth of Staphylococcus aureus on the catheter.

The alcohol can be, for example, ethanol, or one or more of ethanol, propanol, isopropanol and butanol. The solution containing alcohol can further comprise an anti-coagulant, such as, for example, heparin.

The invention also provides an implantable catheter that can be disinfected in vivo with alcohol, wherein the catheter comprises a lumen with an inner surface that is impregnated or coated with chlorhexidine and wherein chlorhexidine is present in the catheter in a concentration that is subinhibitory for attachment or growth of microorganisms on the catheter.

When the implantable catheter is implanted in a subject and not in use, the catheter can be filled with a solution containing alcohol and then capped off or “locked.” The alcohol lock can contain one or more anticoagulants. The alcohol lock can be left in place as long as the catheter is not in use or for a period of time specified by a physician. The invention provides a method of disinfecting or preventing infection of a catheter implanted in a subject, the method comprising flushing a lumen of the catheter with a solution comprising alcohol, wherein at least an inner surface of the lumen is impregnated or coated with chlorhexidine.

The catheter can be disinfected in vivo with a solution comprising 5-100% alcohol, preferably 25-70% alcohol, more preferably 25-50% alcohol, and still more preferably 25-30% alcohol. The catheter can be disinfected in vivo with a solution comprising alcohol at a concentration that, in the absence of chlorhexidine, is subinhibitory for attachment or growth of microorganisms on the catheter.

The chlorhexidine in the catheter can be in the form of chlorhexidine base and/or a chlorhexidine salt. Chlorhexidine salts include, for example, chlorhexidine diphosphanilate, chlorhexidine digluconate, chlorhexidine diacetate, chlorhexidine dihydrochloride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-alpha-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxynaphthoate, and chlorhexidine embonate. Preferred forms of chlorhexidine include chlorhexidine base and chlorhexidine succinate.

Chlorhexidine can be present in the catheter in a concentration of 0.1%-10% by weight of the catheter and preferably 0.5%-5% by weight of the catheter. The chlorhexidine can have a concentration, for example, of about 200 micrograms per cm length of catheter.

Preferably, with the implantable catheters and methods of the present invention, growth of one or more of fungal, gram positive or gram negative pathogenic microorganisms including Candida albicans, Staphylococcus aureus, or Pseudomonas aeruginosa is inhibited. Preferably, the radius of the zone of inhibition (ZOI) of microorganisms, such as e.g. Candida albicans, around the catheter is increased by at least 50% in the presence of 5% ethanol, compared to the radius of the ZOI in the absence of ethanol. More preferably, the radius of the zone of inhibition (ZOI) of microorganisms is at least doubled in the presence of 5% ethanol, compared to the radius of the ZOI in the absence of ethanol. The zone of inhibition can be determined, for example, as set forth herein in the Examples.

Chlorhexidine can be the only anti-infective agent impregnated in or coated on the catheter. However, in addition to chlorhexidine, the catheter can also be impregnated with or coated with a substance selected from the group consisting of one or more of minocycline, triclosan, ethylene diamine tetraacetic acid (EDTA), citrate, taurolidine, 5-Fluorouracil, miconazole, ketoconazole, chlorohexidine and itraconazole.

Chlorhexidine can be used in combination with an antimicrobial dye. Examples of such combination include gendine, genlenol, genlosan and genfoctol (e.g., US 2003/0078242 A1, US 2005/0197634 A1).

The catheter can be implanted in a subject, for example in a vessel such as a blood vessel or in a body cavity. Examples of such catheters include transcutaneous catheters; vascular catheters including peripheral catheters, central catheters, venous catheters, and arterial catheters; urinary catheters; and dialysis catheters.

The present invention is illustrated in the following Experimental Details section, which is set forth to aid in the understanding of the invention, and should not be construed to limit in any way the scope of the invention as defined in the claims that follow thereafter.

### Experimental Details

Determinations of Minimum Inhibitory Concentration (MIC) and Minimum Bacteriocidal Concentration (MBC) were performed using standard microbiological techniques. Chlorhexidine diacetate was tested at the following μg/mL concentrations: 256, 128, 64, 32, 16, 8, 4, 2, 1, 0.5, 0.25, and 0.125. Ethanol was tested at the following percent (%) concentrations: 50, 40, 30, 25, 20, 15, 12, 10, 7, and 5. Dilutions were prepared in Cationic Adjusted Mueller Hinton Broth (CAMHB). All tests were performed in microtiter plates with a total test volume of 100 μL, including diluted antimicrobial agent and inoculum. Concentrations of antimicrobial agents were prepared at 2× concentration, and 50 μL was added to each well of a microtiter plate, to which 50 μL of inoculum of the test organism was added to give the target test concentration.

Inoculum was prepared by transfer of isolated colonies from agar plates to 10 mL of trypticase soy broth (TSB), and incubation at 37° C. for 4 hrs. Cells were subsequently centrifuged, and resuspended twice in phosphate buffered saline. The population of this bacterial suspension was determined by reading optical density at 670 nm. Final concentrations of inoculum were adjusted to a range of approximately 5×10⁴ cfu/mL, using CAMHB as a diluent. Initial concentrations were verified using the Miles and Misra drop count method. To each test well of a microtiter plate, 50 μl of inoculum was added along with 50 μl of the 2× concentration of the antimicrobial agent being tested. Incubation was at 37° C. for 16 to 20 hours.

The MIC was the lowest concentration of antimicrobial agent that completely inhibited growth of the organism in the micro dilution wells as detected by the unaided eye. To determine MBC values, 50 μl was removed from each well showing inhibition of growth (MIC and greater). Solutions from these inhibitory concentrations of antimicrobial agent were diluted in CAMHB to concentrations where they would no longer inhibit the growth of the test organism. Recovery counts were obtained by plating aliquots of the diluted material onto TSA plate containing 5% sheep's blood, and incubating 24 hours at 37° C. The MBC value was calculated as the concentration where the colony forming units equal 1% of those found at the MIC.

Minimum Inhibitory (MIC) and Minimum Bacteriocidal (MBC) ethanol concentrations measured for a variety of common pathogens values are reported in Table 1. These results suggest a lock at ethanol concentrations above 25% will be cidal towards most commonly encountered infectious pathogens. At concentrations below 7% the solution will not inhibit microbial growth.

TABLE 1
Minimum Inhibitory Concentration (MIC) and Minimum Bacteriocidal
Concentration (MBC) for ethanol for a variety of common pathogens.

| Organism | MIC | MBC |
|---|---|---|
| P. aeruginosa ATCC 27853 | 7% | 12% |
| E. cloacae ATCC 13047 | 7% | 10% |
| E. faecalis ATCC 51299 | 7% | 10% |
| C. albicans ATCC 10231 | 7% | 10% |
| K. pneumoniae ATCC 10031 | 10% | 25% |
| E. coli ATCC 25922 | 10% | 15% |
| S. aureus ATCC 29213 | 12% | 25% |
| S. epidermidis ATCC 35983 | 12% | 10-12% |

ATCC, American Type Culture Collection, Manassas, Virginia

The synergy of ethanol and chlorhexidine is illustrated in Table 2 by measurement of the minimum bacteriocidal concentration (MBC) of chlorhexidine at ethanol concentrations that are subinhibitory (80% of the ethanol minimum inhibitory concentration). The chlorhexidine MBC drops (hence potency is increased) by approximately 50% in the presence of subinhibitory ethanol concentrations.

TABLE 2
Minimum Bacteriocidal Concentration (MBC) for chlorhexidine
diacetate (CHA) at 80% of the ethanol minimum inhibitory
concentration.

| | | | MBC for CHA |
|---|---|---|---|
| | MBC for CHA | Ethanol % | @ 80% MIC ETOH |
| Organism | (μg/mL) | (80% of MIC) | (μg/mL) |
| E. faecalis | 8 | 5.6% | 4 |
| S. aureus | 1-2 | 9.6% | 1 |
| E. coli | 4 | 8% | 2 |

5Fr Catheter segments were bulk impregnated with chlorhexidine base (CHX) by soaking in an ethyl acetate solution of CHX and then evaporating the solvent. The chlorhexidine mass per unit length was determined by dissolving the catheter in and then extracting the chlorhexidine. The measure mass of chlorhexidine was approximately 200 micrograms per cm. 5Fr catheter segments were similarly impregnated with chlorhexidine diacetate to a level of 200 micrograms/cm by soaking in an ethyl acetate-methanol solution followed by evaporation. These segments were used in Zone of inhibition testing and release kinetics testing.

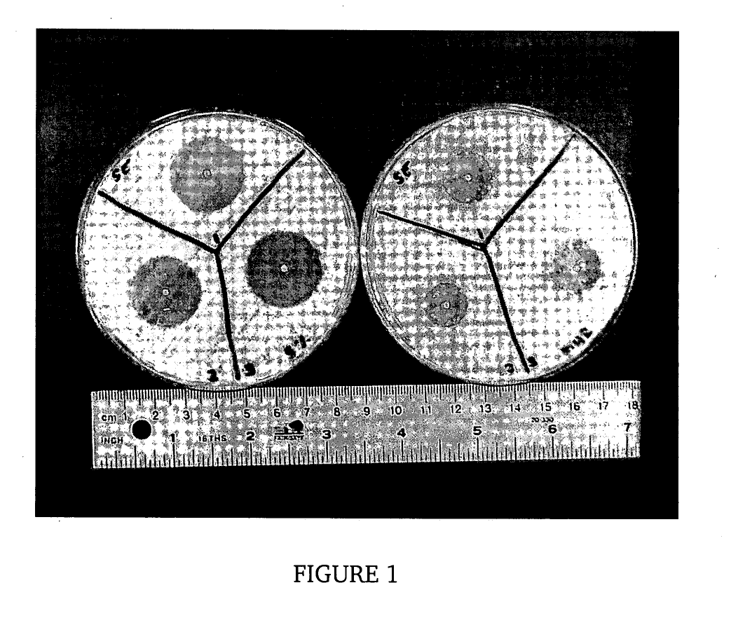

Zones of inhibition (ZOI) against Candida albicans was measured by a modified Kirby Bauer method. Inoculum was prepared by inoculating trypticase soy broth (TSB) with isolated colonies from Yeast Malt Agar (YMA), and incubating the broth at 37° C. overnight. YMA plates with and without 5% ethanol were utilized for ZOI testing. To achieve 5% ethanol, medical grade ethanol (100%) was added to YMA post autoclaving. The media was compounded with 5% less water, and this volume was provided by adding the ethanol just prior to pouring the plates. Plates were swab inoculated with 100 μL of inoculum. Following inoculation, treated catheter segments were inserted vertically into the agar, allowing adequate space between segments to avoid overlap of ZOIs. Plates were. incubated at 37° C. for 24 hours. Zones were measured with calibrated calipers and represent the diameter of the circular ZOI surrounding the treated catheter material. The catheters were transferred every 24 hours to a fresh plate that was also inoculated as described above. The extent of protection against surface interaction with Candida albicans is tabulated below in Table 3, where ZOI is the zone of inhibition measured in millimeters. Zones of inhibition were enhanced for the CHX impregnated catheter in the presence of 5% ethanol. A picture of the enhanced Zone of Inhibition for the catheter segments in the presence of 5% ethanol at 24 hours is shown in FIG. 1.

TABLE 3
Zone of inhibition (ZOI) against Candida albicans for catheter
impregnated with chlorhexidine base in the presence or absence
of 5% ethanol.

| | ZOI (mm) | ZOI (mm) |
|---|---|---|
| Day | (no ethanol) | (with 5% ethanol) |
| 1 | 7 | 12 |
| 2 | 5 | 9 |
| 3 | 4 | 9 |
| 4 | 4 | 9 |
| 5 | 0 | 8 |
| 6 | 2 | 7 |
| 7 | 0 | 6 |
| 14 | 0 | 4 |
| 21 | 0 | 4 |
| 24 | 0 | 0 |

Release kinetics measurements were performed of chlorhexidine base (CHX) and chlorhexidine diacetate (CHA) loaded catheter segments (200 micrograms/cm) in water and in 25% ethanol lock. One cm long catheter segments were placed in 10 ml of 25% ethanol lock solution. At each sampling point the eluting solution was removed and replaced with fresh solution. The amount of chlorhexidine released was measured by UV absorbance readings at 263 nm on the eluting lock solution relative to a reference curve of chlorhexidine standards prepared to known concentrations in 25% ethanol lock. Cumulative % release was calculated from the measured amount released at each time point and the quantity of chlorhexidine loaded into the catheter segments.

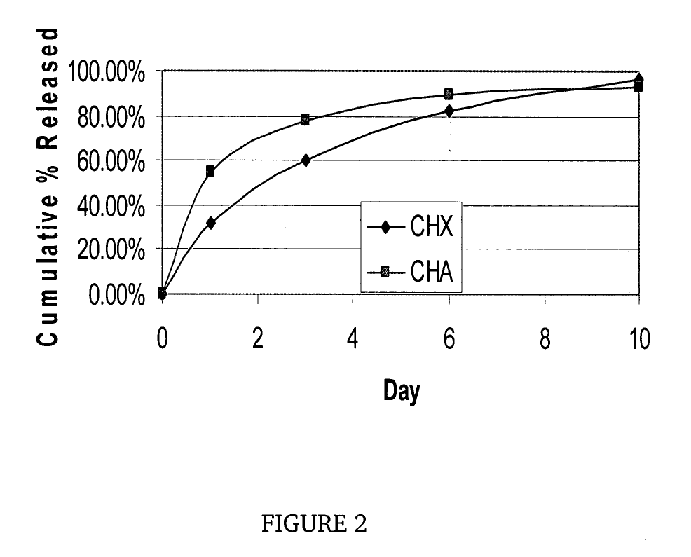

The release curve (FIG. 2) shows that the chlorhexidine diacetate releases faster into the 25% ethanol lock solution than does chlorhexidine base. Thus, chlorhexidine base would be a preferred form of chlorhexidine in this invention. The invention is still viable however with chlorhexidine diacetate. Other less ethanol soluble forms of chlorhexidine would also be preferred over chlorhexidine diacetate and provide longer, duration of activity. The range of ethanol in the lock in this invention can be 5-100%. A preferred range is 25-30%.

Table 4 shows the solubility of different chlorhexidine salts in water and 100% ethanol. Chlorhexidine succinate is a preferred form of chlorhexidine due to its low solubility in alcohol.

TABLE 4
Solubility of different chlorhexidine salts in ethanol (100%) and water.

| Chlorhexidine Salt | Ethanol Solubility | Water Solubility |
|---|---|---|
| Glycolate | 100 mg/ml | 200 mg/ml |
| Laurate | 200 mg/ml | 0.01 mg/ml |
| Benzoate | 33 mg/ml | 0.45 mg/ml |
| Succinate | 0.09 mg/ml | 0.25 mg/ml |
| Propionate | 100 mg/ml | 3.2 mg/ml |
| Valerate | 20 mg/ml | 1.2 mg/ml |

### Discussion

The present invention relates to medical devices, preferably intravenous catheters, with chlorhexidine (and or its salts) incorporated in the device either as a coating or bulk distributed used in synergistic combination with an alcohol lock solution to inhibit attachment and/or growth of microorganisms, thereby preventing device-related infection. Addition of chlorhexidine directly into the lock solution can be undesirable in that if the lock is inadvertently flushed into a patient's bloodstream the individual will be exposed to a circulating bolus of chlorhexidine and ethanol with potential for detrimental toxic side effects. These include enhanced local irritation and necrosis over ethanol alone. Since the microbial attachment and colonization associated with catheter-related bloodstream infections occurs at the surface of the device, the inventors have surprisingly found that sufficiently high surface concentrations of chlorhexidine and ethanol can be attained such that they are bacteriocidal against common infectious pathogens with reduced risk of toxicity resulting from lower systemic exposure to cytotoxic concentrations of ethanol and chlorhexidine. Sublethal ethanol concentrations can occur at the tip of the catheter by leakage or diffusion of ethanol into the blood stream. The inventors have surprisingly found that even if the ethanol concentration drops to just 5% (a sublethal ethanol level), chlorhexidine potency is increased substantially. If chlorhexidine were directly dissolved in the lock solution, leakage and diffusion of both chlorhexidine and ethanol from the catheter tip could result in local levels (at the tip) insufficient to kill potential infectious pathogens. Surprisingly, chlorhexidine can be beneficially released from the surface of the catheter at sufficiently high local concentrations to substantially enhance the potency of the combination (even at sublethal ethanol concentrations) against highly recalcitrant pathogens such as Candida yeast species. The inventors have further found that it is desirable to utilize less alcohol soluble forms of chlorhexidine (such as chlorhexidine base) than more alcohol soluble forms (such as chlorhexidine diacetate) as this decreases the quantity of chlorhexidine extracted during exposure to the lock solution thereby increasing the longevity of surface protection through repeated alcohol locking cycles. This invention allows the effective use of lower ethanol concentrations. Clinical usage of ethanol lock solutions has ranged from concentrations of 25% to 100%. More effective use of ethanol concentrations at the low end of this range (towards 25%) enhances safety to the patient from two standpoints. One is the previously mentioned reduced risk of toxic side effects due to exposure of tissues to high local ethanol concentrations, and the second is reduced risk of compromise of catheter mechanical strength due to prolonged exposure of catheter materials to higher ethanol concentrations. In addition to chlorhexidine, other antimicrobial agents (such as antiseptics, antibiotics, chemotherapeutics and antimycotics or combinations) can be incorporated into catheters to enhance rates of inhibition and lethality towards pathogens at catheter surfaces in combination with an ethanol lock. Of special note are minocycline, triclosan, ethylene diamine tetraacetic acid (EDTA), citrate, taurolidine, 5-Fluorouracil, miconazole, ketoconazole, chlorohexidine and itraconazole.

### REFERENCES

Ball P A, Brokenshire E, Parry B, Merrie A, Gillanders L, McIlroy K, Plank L. Ethanol locking as a possible treatment for microbial contamination of long-term central venous catheters. Nutrition 2003; 19:570.

Bestul et al. Antibiotic Lock Technique: Review of the Literature. Pharmacotherapy. 2005;25 (2):211-227.

Crnich C J, Halfmann J A, Crone W C, Maki D G. The effects of prolonged ethanol exposure on the mechanical properties of polyurethane and silicone catheters used for intravascular access. Infect Control Hosp Epidemiol. 2005 August;26(8):708-14.

Dannenberg C, Bierbach U, Rothe A, Beer J, Korholz D. Ethanol-lock technique in the treatment of bloodstream infections in pediatric oncology patients with brovaic catheter. J Pediatr Hematol Oncol 2003; 25(8):616-21.

Denton G W. Chlorhexidine. In: Seymour S. Block (Ed.) Disinfection, sterilization, and preservation. 4th Ed., Lea & Febiger, Williams & Wilkins, Media PA, 1991:279. 2.

Mecalf S C, Chambers S T, Pithie A D. Use of ethanol locks to prevent recurrent central line sepsis. J Infect. 2004; 49:20-2.

O'Grady et al. Guidelines for the Prevention of Intravascular Catheter-Related infections. Clinical Infectious Diseases 2002;35:1281-1307.

Safdar N, Maki D G. The pathogenesis of catheter-related bloodstream infection with noncuffed short-term central venous catheters. Intensive Care Med. 2004 Jan;30(1):62-7. Epub 2003 November 26.

Saint S, Veenstra D L, Lipsky B A. The clinical and economic consequences of nosocomial central venous catheter-related infection: are antimicrobial catheters useful? Infect Control Hosp Epidemiol. 2000 June;21(6):375-80.

Segura et al. A clinical trial on the prevention of catheter-related sepsis using a new hub model. Ann Surg. 1996;223:363-369.

Sissons et al. Inhibition by ethanol of the growth of biofilm and dispersed microcosm dental plaques. Archives of Oral Biology, Vol. 41, 1, JN 1996;27-34.

University of Wisconsin News Release. Ethanol Treatment May Be Instrumental in Fighting IV-Based Infection, http:www.news.wisc.edu/releases/11399.html, Aug. 10, 2005.

PCT International Publication No. WO 95/12395, published May 11, 1995, Topical Antimicrobial Cleanser Containing Chlorhexidine Gluconate And Alcohol, Calgon Vestal Lab., Inc., Osborne et al.

U.S. Patent Application Publication No. US 2003/0078242 A1, published Apr. 24, 2003, Raad et al., Novel antiseptic derivatives with broad spectrum antimicrobial activity for the impregnation of surfaces.

U.S. Patent Application Publication No. US 2005/0197634A1, published Sep. 8, 2005, Raad et al., Methods for coating and impregnating medical devices with antiseptic compositions.

U.S. Pat. No. 5,017,617, issued May 21, 1991, Kihara et al., Disinfectant composition for medical use.

U.S. Pat. No. 5,019,096, issued May 28, 1991, Fox, Jr. et al., Infection-resistant compositions, medical devices and surfaces and methods for preparing and using same.

U.S. Pat. No. 5,443,385, issued Aug. 25, 1995, Overmyer, Method of disinfecting and lubricating dental/medical device.

U.S. Pat. No. 5,451,424, issued Sep. 19, 1995, Solomon et al., Anti-infective and antithrombogenic medical articles and method for their preparation.

U.S. Pat. No. 5,707,366, issued Jan. 13, 1998, Solomon et al., Anti-infective and antithrombogenic medical articles and method for their preparation.

U.S. Pat. No. 5,753,246, issued May 19, 1998, Peters, Packaged germicidal towelette, sanitation kit and method for promoting hygiene.

U.S. Pat. No. 5,776,430, issued Jul. 7, 1998, Osborne et al., Topical antimicrobial cleanser containing chlorhexidine gluconate and alcohol.

U.S. Pat. No. 5,800,827, issued Sep. 1, 1998, Igarashi et al., Disinfectant composition.

U.S. Pat. No. 5,906,808, issued May 25, 1999, Osborne et al., Process of preparing a topical antimicrobial cleanser.

U.S. Pat. No. 6,261,271, issued Jul. 17, 2001, Solomon et al., Anti-infective and antithrombogenic medical articles and method for their preparation.

U.S. Pat. No. 6,679,870 B1, issued Jan. 20, 2004, Finch et al., Methods and kits for locking and disinfecting implanted catheters.

U.S. Pat. No. 6,685,694 B2, issued Feb. 3, 2004, Finch et al., Methods and kits for locking and disinfecting implanted catheters.

U.S. Pat. No. 6,869,623, issued Mar. 22, 2005, Viamonte et al., Non-toxic mucosal disinfectant containing isopropyl alcohol, sesame oil, aloe, and lemon oil.

1. An implantable catheter comprising a lumen that is at least partly filled with a solution comprising alcohol, wherein at least an inner surface of the lumen is impregnated or coated with chlorhexidine.

2. The implantable catheter of claim 1, wherein the catheter is impregnated with chlorhexidine.

3. The implantable catheter of claim 1, wherein an inner surface of the lumen is coated with chlorhexidine.

4. The implantable catheter of claim 1, wherein chlorhexidine is present in a concentration of 0.1%-10% by weight of the catheter.

5. The implantable catheter of claim 1, wherein chlorhexidine is present in a concentration of 0.5%-5% by weight of the catheter.

6. The implantable catheter of claim 1, wherein chlorhexidine has a concentration of about 200 micrograms per cm length of catheter.

7. The implantable catheter of claim 1, wherein alcohol is present in the solution in a concentration of 25-30%.

8. The implantable catheter of claim 1, wherein chlorhexidine is present in a concentration that, in the absence of alcohol, is subinhibitory for attachment or growth of microorganisms on the catheter.

9. The implantable catheter of claim 1, wherein alcohol is present in a concentration that, in the absence of chlorhexidine, is subinhibitory for attachment or growth of microorganisms on the catheter.

10. The implantable catheter of claim 1, wherein chlorhexidine is present in a concentration that, in the absence of alcohol, is subinhibitory for attachment or growth of microorganisms on the catheter, and wherein alcohol is present in a concentration that, in the absence of chlorhexidine, is subinhibitory for attachment or growth of microorganisms on the catheter.

11. The implantable catheter of claim 8, wherein chlorhexidine is present in a concentration that is subinhibitory for attachment or growth of Staphylococcus aureus on the catheter.

12. The implantable catheter of claim 9, wherein alcohol is present in a concentration that is subinhibitory for attachment or growth of Staphylococcus aureus on the catheter.

13. The implantable catheter of claim 1, wherein chlorhexidine comprises chlorhexidine base.

14. The implantable catheter of claim 1, wherein chlorhexidine comprises chlorhexidine salt.

15. The implantable catheter of claim 1, wherein chlorhexidine comprises chlorhexidine succinate.

16. The implantable catheter of claim 1, wherein at least an inner surface of the lumen is impregnated or coated with a combination of chlorhexidine and an antimicrobial dye.

17. The implantable catheter of claim 16, wherein at least an inner surface of the lumen is impregnated or coated with gendine, genlenol, genlosan, or genfoctol.

18. The implantable catheter of claim 1, wherein the catheter is further impregnated with or coated with a substance selected from the group consisting of one or more of minocycline, triclosan, ethylene diamine tetraacetic acid (EDTA), citrate, taurolidine, 5-Fluorouracil, miconazole, ketoconazole, chlorohexidine and itraconazole.

19. The implantable catheter of claim 1, wherein the alcohol is selected from the group consisting of one or more of ethanol, propanol, isopropanol and butanol.

20. The implantable catheter of claim 1, wherein the alcohol is ethanol.

21. The implantable catheter of claim 1, wherein the solution further comprises an anti-coagulant.

22. The implantable catheter of claim 1, wherein the catheter is implanted in a subject.

23. The implantable catheter of claim 22, wherein the catheter is implanted in a blood vessel or a body cavity.

24. The implantable catheter of claim 1, wherein the catheter is a transcutaneous catheter.

25. An implantable catheter that can be disinfected in vivo with alcohol, wherein the catheter comprises a lumen with an inner surface that is impregnated or coated with chlorhexidine and wherein chlorhexidine is present in the catheter in a concentration that is subinhibitory for attachment or growth of microorganisms on the catheter.

26-41. (canceled)

42. A method of disinfecting or preventing infection of a catheter implanted in a subject, the method comprising flushing a lumen of the catheter with a solution comprising alcohol, wherein at least an inner surface of the lumen is impregnated or coated with chlorhexidine.

43-64. (canceled)
